(19) United States
(12) Patent Application Publication
(10) Pub. No.: US 2025/0114646 A1
(43) Pub. Date: Apr. 10, 2025

(54) SELF-POSITIONING ACOUSTIC LENS

(71) Applicants: INSTITUT NATIONAL DE LA SANTÉ ET DE LA RECHERCHE MÉDICALE, Paris (FR); CENTRE NATIONAL DE LA RECHERCHE SCIENTIFIQUE, Paris (FR); ECOLE SUPÉRIEURE DE PHYSIQUE ET DE CHIMIE INDUSTRIELLES DE LA VILLE DE PARIS, Paris (FR)

(72) Inventors: Jean-François AUBRY, Paris (FR); Thomas TIENNOT, Paris (FR); Mickael TANTER, Paris (FR)

(21) Appl. No.: 18/865,879

(22) PCT Filed: May 15, 2023
(86) PCT No.: PCT/EP2023/063032

§ 371 Date: Nov. 14, 2024

(30) Foreign Application Priority Data
May 16, 2022 (EP) 22305724.1

(51) Int. Cl. A61N 7/02; A61N 7/00

(52) U.S. Cl. CPC A61N 7/022; A61N 2007/006

(57) ABSTRACT

Nowadays, the interest to use ultrasound waves in medical field is well established. Such ultrasound waves may be focused for treating a zone in an organ such as the brain for instance. The focus allows to treat only the zone relative to the disease and avoid treating a healthy zone. Therefore, it is mandatory to use an image guidance system to monitor in real time where the ultrasound waves are focused. The guidance can be performed by Magnetic Resonance Imaging (MRI), Ultrasound Imaging (echography), or Optical Imaging (neuronavigation). However, such systems increase the cost and the complexity of the whole process. The present disclosure overcomes the above drawbacks by proposing a self-positioning acoustic lens allowing to ensure good ultrasound waves transmission in the zone to treat without requiring to the use of a navigation system.

### TECHNICAL FIELD

The present disclosure relates to a self-positioning acoustic lens configured to obtaining a predetermined field of ultrasonic waves in a substantially homogeneous medium masked by a bony structure, and production methods for implementing such self-positioning acoustic lens for these purposes.

### BACKGROUND ART

Nowadays, the interest to use ultrasound waves in the medical field no longer needs to be proven.

Generally, ultrasound may be generated from a probe comprising one or a plurality of transducers able to generate individually ultrasound waves. Such ultrasound waves may be transmitted toward a medium in order to generate in response backscattered signals, which may be acquired and used to generate a 3D ultrasound image for instance.

Another use of such ultrasound wave may be in applications for therapeutic purposes for instance. Indeed, such ultrasound waves may be focused towards an organ (e.g. brain or heart, liver, etc.) in order to treat a specific zone of the organ. For instance, in the case of the brain, this specific zone to treat may be relative to a disease such as Essential tremor or Glioblastoma or Depression. The focusing may be performed in different ways. For instance, when using a plurality of transducers, the focusing may be performed by adjusting the delay on each transmitted ultrasound wave in order to obtain a focal spot at the targeted area. When using only one transducer or few transducers (less than 128 transducers for instance), the focal spot of the ultrasound waves depends on the shape of the ultrasound probe or the shape of the ultrasound transducers (typically the active surface is distributed on a spherical surface). The main advantage to use only one transducer or few transducers (less than 128 transducers for instance) may be the cost reduction and the simplification of the system used to perform the insonification of the medium compared to an equipment doing the same but with a plurality of transducers (superior to 100). However, the inconvenient, when using only one transducer or few transducers for focusing the ultrasound waves, may be the deformation of the focal spot in the area to treat in the medium. Such deformation may happen when the ultrasound waves have to cross a barrier such as bones before reaching the area to treat in the medium. The document WO2017001781A1 describes a method for designing and fabricating an ultrasound lens suitable to focus ultrasound waves in a medium as a brain (i.e. located behind the skull bone barrier) while limiting the deformation of the focal spot.

Besides, when focusing ultrasound wave towards a specific zone of the medium as a specific zone of a brain for instance, it is usually mandatory to use a navigation system allowing to know, in real-time, the position of the ultrasound probe in regard to the surface of the medium. Without such navigation system, the focus could be located in a wrong zone of the brain and damage healthy tissues. For example, a Magnetic Resonance Imaging (MRI) system can be used to locate the transducer and guide the treatment [Elias, W. Jeffrey, et al. “A randomized trial of focused ultrasound thalamotomy for essential tremor.” New England Journal of Medicine 375.8 (2016): 730-739.]. Nevertheless, such systems are expensive, and the treatment must be performed inside the MRI system. One way to decrease the cost of a navigated treatment is to use a neuronavigation system taking advantage of an optical or a magnetic position tracking system. Neuronavigation systems present, however, several drawbacks. One of the main drawback is the need to pre-configure such a system each time a patient needs to be treated. Indeed, before using a neuronavigation system with a patient, the system has to be configured by providing one or several images of the head of the patient in order to get a referential of the brain (or brain cavity) as well as a referential of the surface of the skull. The images may be performed by using, for instance, Magnetic Resonance Imaging, or CT Scan or Ultrasound Imaging. Then reference tools need to be used to locate anatomical landmarks (for example by pointing the nose bridge, or the left and right tragus, or by palping the skin surface) and register the patient head. This pre-configuration has to be carried out at the beginning of each treatment for every patient, which may represent a lost of time for the clinicians. Moreover, neuronavigation systems have a significant cost and not all the clinical departments possess such systems. Besides, medical office and home use of focused ultrasound would benefit from avoiding the use of a neuronavigation system.

Therefore, there is a need to allow the focus of ultrasound waves in a specific zone of a human brain without requiring the use of a neuronavigation system.

### SUMMARY

To this end, the present disclosure proposes a self-positioning acoustic lens comprising a front surface and back surface, said back surface being opposed to the front surface, said self-positioning acoustic lens being adapted for transmitting an ultrasound wave into a medium comprising at least one aberrating barrier and a substantially homogeneous internal part masked by said aberrating barrier, said ultrasound wave being generated by an ultrasound probe located outside of the medium and said back surface facing the emission surface of the ultrasound probe,
wherein the self-positioning acoustic lens being configured to, when the self-positioning acoustic lens is interposed between the ultrasound probe and the aberrating barrier and when the ultrasound probe transmits a predetermined ultrasonic wave, generate a predetermined objective ultrasonic wave field in at least one predetermined area belonging to said internal part despite the presence of the aberrating barrier,
and wherein the front surface may be constrained and may be adapted to match the outer surface of the medium, said outer surface of the medium not being perfectly spherical, and wherein the back surface may be spaced from the front surface according to a specific acoustic lens thickness in order to create the said predetermined objective ultrasonic wave field.

By self-positioning acoustic lens, it is understood that the acoustic lens may be configured to be placed at only one correct possible position on the surface of the medium. The front surface may be constrained and may be adapted to match the outer surface of the medium in order to be self-positioned on the outer surface of the medium. The front surface of the self-positioning acoustic lens may be configured to match the outer surface of the medium in a complementary way at only one correct possible position on the surface of the medium, as one position only will allow a complete contact between the front surface of the lens and the outer surface of the medium.

Thus, advantageously, the self-positioning acoustic lens may serve to treat a zone of interest (or specific zone) comprising in the focal (or focus) spot as previously explained without using any neuronavigation systems. The zone of interest may be a zone located in the brain and related to a disease such as Depression or Essential Tremor for instance. Indeed, thanks to the fact that the front surface is shaped for only one specific area of the surface of the medium (or instance surface of the skin surrounding the skull), the acoustic lens is configured to be placed at only one possible position, therefore self-positioning, on the skull allowing to treat the correct zone (e.g. in the brain) without using any neuronavigation systems. Moreover, the configuration of the self-positioning acoustic lens is such that the acoustic lens thickness compensates the distortions induced by the aberrating medium. As the acoustic lens is designed for a specific zone to treat in the brain from only one possible position on the outer surface or the head, the risk of mistakes (e.g. treating the wrong zone in the brain) is highly minimized.

In one or several embodiments, the surface of contact between the front surface of the self-positioning acoustic lens and the outer surface of the medium may be limited to a restricted and specific area on the outer surface of the medium.

By contact, it may be understood a physical contact between a surface (front surface for instance) of the self-positioning acoustic lens and the outer surface of the medium.

In one or several embodiments, the aberrating barrier is a skull and the outer surface is the skin surrounding the skull, and the restricted and specific area may correspond to a part of the outer surface in contact with the front surface of the self-positioning acoustic lens.

In one or several embodiments, the self-positioning acoustic lens may be made of polydimethylsiloxane, or polymethylpentene or is a compound lens.

In one or several embodiments, the back surface of the self-positioning acoustic lens may be distant from the emission surface of the ultrasound probe according to a value comprised between 0 and 15 centimeters.

In one or several embodiments, the thickness of the self-positioning acoustic lens from each point of the front surface of the self-positioning acoustic lens may satisfy the following formula:

$$e ( x , y , z ) = \frac{Δ t ( x , y , z )}{\frac{1}{C} - \frac{1}{{C}_{1}}} + {e}_{0}$$

where:
c is the speed of the ultrasonic wave in coupling medium outside of the aberrating barrier;
c₁ is the speed of the ultrasonic wave in the self-positioning acoustic lens;
e₀ is a real number such that e(x, y, z) has a value at each point of the self-positioning acoustic lens that is positive and greater than a minimum value necessary to ensure the robustness of the self-positioning acoustic lens, said minimum value depending on the material of the self-positioning acoustic lens;
Δt(x, y, z) is a delay law calculated to recreate the predetermined objective despite the presence of the aberrating medium.

In one or several embodiments, when the ultrasound probe is a focused transducer, the thickness may be defined so that, in spherical coordinates, each point L(r′, θ, φ) of the back surface of the self-positioning acoustic lens in regard to a corresponding point on the front surface M(r, θ, φ), may verify the following formula:

$${r}^{′} = r + \frac{Δ t ( r , θ , φ )}{\frac{1}{C} - \frac{1}{{C}_{1}}} + {e}_{0}$$

where:
c is the speed of the ultrasonic wave in the medium outside of the aberrating barrier;
c₁ is the speed of the ultrasonic wave in the self-positioning acoustic lens;
e₀ is a real number such that the thickness of the self-positioning acoustic lens is positive at each point and greater than a minimum value necessary to ensure the robustness of the self-positioning acoustic lens, said minimum value depending on the material of the self-positioning acoustic lens;
Δt(r, θ, φ) is a delay law calculated to recreate the predetermined objective despite the presence of the aberrating medium, said delay law is calculated at each point M(r, θ, φ) on the front surface defined in the spherical coordinates with the origin of the spherical reference system being located at the center of curvature of the focused transducer.

In one or several embodiments, the front surface may be constrained and may be adapted to match the outer surface of the medium in a complementary way.

The present disclosure also relates to a production method for implementing a self-positioning acoustic lens comprising a front surface and back surface, said back surface being opposed to the front surface, said self-positioning acoustic lens being adapted for transmitting an ultrasound wave into a medium comprising at least one aberrating barrier and a substantially homogeneous internal part masked by said aberrating barrier, the ultrasound wave being generated by an ultrasound probe located outside of the medium and said back surface being adapted to face the emission surface of the ultrasound probe;
the production method may comprise at least:
imaging the medium for generating a mapping of the acoustic properties of the medium;
using a model to estimate time delays at a given control surface comprised within an expected volume of the acoustic lens, said time delays allowing to produce predetermined objective ultrasonic wave field in at least one predetermined area belonging to said internal part;
calculating a geometry of the self-positioning acoustic lens comprising at least a determination of a first shape of front surface and a determination of a thickness between the front and the back surface, by using the model of the medium comprising the mapping of acoustic properties, the self-positioning acoustic lens being configured to generate the time delays so that, when the self-positioning acoustic lens is interposed between the ultrasound probe and the aberrating barrier and when the ultrasound probe transmits a predetermined ultrasonic wave it produces the predetermined objective ultrasonic wave field in at least one predetermined area belonging to said internal part despite a presence of the aberrating barrier;
implementing the self-positioning acoustic lens by using the calculated self-positioning acoustic lens;
and wherein the first shape of the front surface of the self-positioning acoustic lens is determined to match the outer surface of the medium, said outer surface of the medium not being perfectly spherical.

In one or several embodiments, the surface of contact between the front surface of the self-positioning acoustic lens and the outer surface of the medium may be limited to a restricted and specific area on the outer surface of the medium.

By contact, it may be understood a physical contact between a surface (front surface for instance) of the self-positioning acoustic lens and the outer surface of the medium.

In one or several embodiments, the imaging of the medium may be performed by using computed tomography (CT) or Magnetic Resonance Imaging (conventional imaging or Ultra-short echo-time imaging) or Ultrasound Imaging.

In one or several embodiments, the calculation of the geometry of the self-positioning acoustic lens may be preceded by a simulation comprising:
(b1) a back-propagation in the medium of said predetermined objective ultrasonic wave field from the predetermined area to a chosen position of the control surface is simulated;
(b2) first arrival times t₁(x, y, z) of said back-propagation of said predetermined objective ultrasonic wave field to said chosen position of the control surface are determined;
(b3) a propagation in a coupling medium of the ultrasonic wave emitted by the ultrasound probe disposed outside of the medium to the chosen position of the front face of the self-positioning acoustic lens is calculated, and second arrival times t₀(x, y, z) of said ultrasonic wave to said chosen position of the front face of the self-positioning acoustic lens are determined;
(b4) a delay law Δt₀(x, y, z) at said chosen position of the control surface equal to the sum of the first arrival times t₁(x, y, z) and second arrival times t₀(x, y, z) is determined,
wherein the geometry of the self-positioning acoustic lens is calculated using said delay law Δt₀(x, y, z), so that when the ultrasound probe emits the ultrasonic wave through said self-positioning acoustic lens, said predetermined ultrasonic wave reproduces the objective wave field in said predetermined area after passing through said aberrating barrier, and
wherein control points M(x, y, z) distributed on the front surface of the self-positioning acoustic lens and at which points the second arrival times t₀(x, y, z) and the first arrival times t₁(x, y, z) are determined, belong to a single control surface, preferably the control surface is located at the outer surface of the medium.

In one or several embodiments, when the ultrasound probe is a focused transducer, the thickness may be determined so that, in spherical coordinates, each point L(r′, θ, φ) of the back surface of the self-positioning acoustic lens in regard to a corresponding point on the front surface M(r, θ, φ), may be calculated with the following formula:

$${r}^{′} = r + \frac{Δ t ( r , θ , φ )}{\frac{1}{C} - \frac{1}{{C}_{1}}} + {e}_{0}$$

where:
c is the speed of the ultrasonic wave in the coupling medium of the aberrating barrier;
c₁ is the speed of the ultrasonic wave in the self-positioning acoustic lens;
e₀ is a real number such that the thickness of the self-positioning acoustic lens is positive at each point and greater than a minimum value necessary to ensure the robustness of the self-positioning acoustic lens, said minimum value depending on the material of the self-positioning acoustic lens;
Δt₀(r, θ, φ) is a delay law calculated to recreate the predetermined objective despite the presence of the aberrating medium, said delay law is calculated at each point on the front surface M(r, θ, φ) defined in the spherical coordinates with the origin of the spherical reference system being located at the center of curvature of the focused transducer.

In one or several embodiments, if the ultrasonic wave is monochromatic, the thickness may be determined so that, in spherical coordinates, each point L(r′, θ, φ) of the back surface of the self-positioning acoustic lens in regard to a corresponding control point M(r, θ, φ), may be calculated with the following formula:

$${r}^{′} = r + \frac{Δ t ( r , θ , φ )}{\frac{1}{c} - \frac{1}{{c}_{1}}} + {e}_{0} , modulo \frac{T}{\frac{1}{c} - \frac{1}{{c}_{1}}}$$

where:
c is the speed of the ultrasonic wave in the medium outside of the aberrating barrier;
c1 is the speed of the ultrasonic wave in the self-positioning acoustic lens;
e0 is a real number such that the thickness of the self-positioning acoustic lens is positive at each point and greater than a minimum value necessary to ensure the robustness of the self-positioning acoustic lens, said minimum value depending on the material of the self-positioning acoustic lens;
Δt₀(r, θ, φ) is a delay law calculated to recreate the predetermined objective despite the presence of the aberrating medium, said delay law is calculated at each point on the constrained front surface M(r, θ, φ) defined in the spherical coordinates with the origin of the spherical reference system being located at the center of curvature of the focused transducer.

In one or several embodiments, the thickness e(x, y, z) may be calculated at each point M(x, y, z) of the front surface with the following formula:

$$e ( x , y , z ) = \frac{Δ t ( x , y , z )}{\frac{1}{C} - \frac{1}{{C}_{1}}} + {e}_{0}$$

where:
c is the speed of the ultrasonic wave in the coupling medium outside of the aberrating barrier;
c₁ is the speed of the ultrasonic wave in the self-positioning acoustic lens;
e0 is a real number such that the thickness e(x, y, z) of the self-positioning acoustic lens is positive at each point and greater than a minimum value necessary to ensure the robustness of the self-positioning acoustic lens, said minimum value depending on the material of the self-positioning acoustic lens.

In one or several embodiments, if the ultrasonic wave is monochromatic, the thickness e(x, y, z) may be calculated at each point M(x, y, z) of the front with the following formula:

$$e ( x , y , z ) = \frac{Δ t ( x , y , z )}{\frac{1}{c} - \frac{1}{{c}_{1}}} + {e}_{0} , modulo \frac{T}{\frac{1}{c} - \frac{1}{{c}_{1}}}$$

where:
c is the speed of the ultrasonic wave in the coupling medium outside of the aberrating barrier;
c₁ is the speed of the ultrasonic wave in the self-positioning acoustic lens;
e₀ is a real number such that e(x, y, z) has a has a value at each point of the self-positioning acoustic lens that is positive and greater than a minimum value necessary to ensure the robustness of the self-positioning acoustic lens, said minimum value depending on the material of the self-positioning acoustic lens;
Δt₀(x, y, z) is a delay law calculated to recreate the predetermined objective despite the presence of the aberrating medium;
T is the period of the ultrasonic wave.

In one or several embodiments, implementing the self-positioning acoustic lens may be done by a method chosen among three-dimensional printing of the self-positioning acoustic lens and/or digitally controlled machining of at least one block of material for forming the self-positioning acoustic lens.

In one or several embodiments, implementing the self-positioning acoustic lens may comprise at least:
(d1) a mold implementation done by a method chosen among three-dimensional printing of said at least one mold and/or numerically controlled machining of at least one block of material for forming said at least one mold;
(d2) a molding in which said self-positioning acoustic lens or at least one component of said self-positioning acoustic lens is molded in said at least one mold.

In one or several embodiments, the self-positioning acoustic lens may be made of a material having acoustic properties modifiable by exposure to a predetermined radiation,
during the calculation of geometry of the self-positioning acoustic lens, the self-positioning acoustic lens is calculated by taking into account the local acoustic properties in the said self-positioning acoustic lens;
and the method comprises an implementation of the self-positioning acoustic lens during which said self-positioning acoustic lens is locally exposed to said predetermined radiation for obtaining the local acoustical properties of the self-positioning acoustic lens needed to generate the calculated time delay law Δt₀(x, y, z) needed to recreate the predetermined objective despite the presence of the aberrating medium.

In one or several embodiments, the first shape of the front surface of the self-positioning acoustic lens may be determined to match the outer surface of the medium in a complementary way.

In one or several embodiments, the calculation of geometry of the self-positioning acoustic lens may be done iteratively in order to optimize the ultrasonic energy transmission yield in the predetermined zone, using an analytic or numerical model.

In one or several embodiments, at sub-step (b3), the second arrival times t₀(x, y, z) may be determined by simulating propagation of said predetermined ultrasonic wave in a simplified model allowing an analytic calculation.

In one or several embodiments, the calculation of geometry of the self-positioning acoustic lens may consider the travel times of said predetermined ultrasonic wave in the self-positioning acoustic lens and angles of incidence and refraction of said predetermined ultrasonic wave at the surface of the self-positioning acoustic lens.

In one or several embodiments, the calculation of geometry of the self-positioning acoustic lens may consider the entire propagation of said predetermined ultrasonic wave, including refraction by each surface of the self-positioning acoustic lens and/or echoes between the ultrasound probe, the self-positioning acoustic lens and the aberrating barrier and/or including echoes within the self-positioning acoustic lens.

The present disclosure also relates to an insonification method of a medium comprising at least one aberrating barrier and a substantially homogeneous internal part masked by said aberrating barrier, said outer surface of the medium not being perfectly spherical, the insonification method may comprise a manufacturing of a self-positioning acoustic lens according to the present disclosure;
the insonification method further may comprise:
transmitting said predetermined ultrasonic wave through the self-positioning acoustic lens, the ultrasound wave being generated by said ultrasound probe located outside of the medium and said back surface being adapted to face the emission surface of the ultrasound probe,
and wherein the self-positioning acoustic lens being disposed on the outer surface of the medium so that the first shape of the front surface of the self-positioning acoustic lens match with the outer surface of the medium.

In one or several embodiments, the surface of contact between the front surface of the self-positioning acoustic lens and the outer surface of the medium may be limited to a restricted and specific area on the outer surface of the medium.

By contact, it may be understood a physical contact between a surface (the front surface for instance) of the self-positioning acoustic lens and the outer surface of the medium.

In one or several embodiments, the medium in which the ultrasonic waves propagate may be a human or animal head, where the aberrating barrier may be a skull and the internal part may be a brain.

In one or several embodiments, the predetermined ultrasonic wave field may be focused in said internal part in at least one predetermined zone.

The present disclosure also relates to a method for calculating a geometry of a self-positioning acoustic lens, the self-positioning acoustic lens being suitable for insonification of a medium comprising at least one aberrating barrier and a substantially homogeneous internal part masked by said aberrating barrier, the outer surface of the medium not being perfectly spherical,
the method for calculating the geometry of the self-positioning acoustic lens, by using a model of the medium comprising a mapping of acoustic properties, the self-positioning acoustic lens being configured to, when the self-positioning acoustic lens is interposed between the ultrasound probe and the aberrating barrier and when the ultrasound probe transmits a predetermined ultrasonic wave, generates a predetermined objective ultrasonic wave field in at least one predetermined area belonging to said internal part.

The present disclosure also relates to a calculation device of a geometry of a self-positioning acoustic lens, the self-positioning acoustic lens being suitable for insonification of a medium comprising at least one aberrating barrier and a substantially homogeneous internal part masked by said aberrating barrier, the outer surface of the medium not being perfectly spherical,
the calculation device being configured to calculate said geometry of the self-positioning acoustic lens, using a model of the medium comprising the mapping of acoustic properties, the self-positioning acoustic lens being configured to, when the self-positioning acoustic lens is interposed between the ultrasound probe and the aberrating barrier and when the ultrasound probe transmits a predetermined ultrasonic wave, generates a predetermined objective ultrasonic wave field in at least one predetermined area belonging to said internal part.

The present disclosure also relates to a device for manufacturing a self-positioning acoustic lens, comprising a calculation device of a self-positioning acoustic lens according to the present disclosure and means for producing the self-positioning acoustic lens.

The present disclosure also relates to a computer program comprising instructions which, when the program is executed by a computer comprising a processor and a storage, cause the computer to carry out the methods of the present disclosure.

The present disclosure also relates to a computer-readable medium having stored thereon the computer program of the present disclosure.

The present disclosure also relates to a non-transitory computer readable medium having stored thereon software instructions that, when executed by a processor, cause the processor to carried out the methods of the present disclosure.

### BRIEF DESCRIPTION OF DRAWINGS

Other features, details and advantages will be shown in the following detailed description and on the figures, on which:

FIG. 1 & FIG. 2 schematically illustrate an overall view of an ultrasonic wave generation mechanism according to the present disclosure, comprising an acoustic lens.

FIG. 3 illustrates the method for obtaining an acoustic lens such the acoustic lens of the present disclosure.

FIG. 4 is a drawing illustrating the operation of the mechanism from FIG. 1.

FIG. 5a illustrates a predetermined ultrasound wave field in an area which is not corrected.

FIG. 5b illustrates a predetermined ultrasound wave field in the same area corrected with a self-positioning acoustic lens.

### DESCRIPTION OF EMBODIMENTS

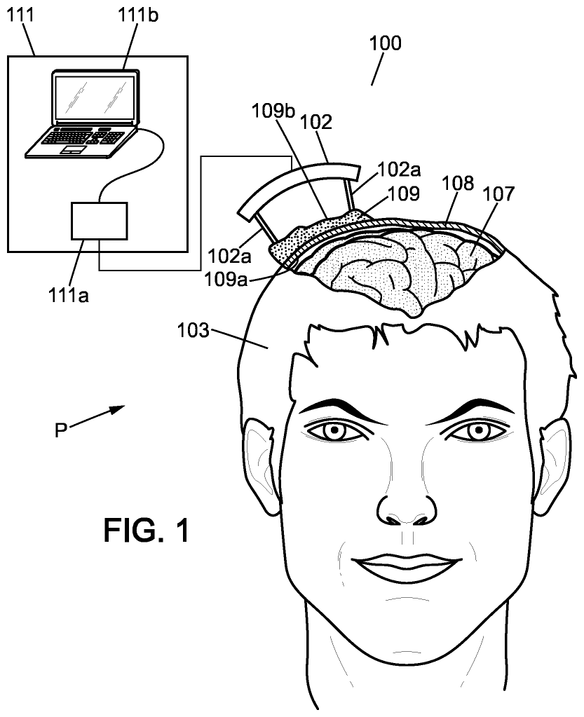

FIG. 1 and FIG. 2 schematically illustrate an overall view of an ultrasonic wave generation mechanism according to the present disclosure, comprising a self-positioning acoustic lens.

In the various figures, the same references designate identical or similar items.

The mechanism 100 for generation of ultrasonic wave(s) shown on FIG. 1 may be capable of generating predetermined ultrasonic waves fields (or ultrasonic waves) in a medium 103, for example the head of a patient P. The medium 103 may comprise at least one aberrating barrier 108 and at least one substantially homogeneous internal part 107 masked by said aberrating barrier 108.

According to an example, the aberrating barrier may be the skull of the patient P and the internal part may be the brain of the patient P.

The mechanism 100 may be intended to generate ultrasonic waves in the brain 107 of the patient P (or more generally in the internal part 107 of the medium 103) at frequencies for example in the range of 0.1 to 10 MHZ, in particular from 0.2 to 3 MHz, from the outside of the patient P.

This generation of ultrasonic waves may be intended for example to treat a zone of the brain which may be associated to a specific pathology as Parkinson, Essential Tremor, Depression, Anxiety, Schizophrenia, Alzheimer's Disease or other psychiatric or neurological disorders. The treatment could for example consist in ultrasound thermal ablation, ultrasound neuromodulation, ultrasound histotripsy, or ultrasound blood brain barrier opening for drug delivery.

In these applications, it should be noted that obtaining an objective wave field is never in itself a therapeutic treatment, but a simple technical measure for focusing waves. The possible therapeutic treatment, selected by a physician, consists in the choice of the objective wave field, the intensity thereof, the duration of the application thereof, the number of applications of this objective wave field and their distribution over time.

By objective wave field, it may be understood a wave field focused (or focus) on a focal point in the brain 107 for instance.

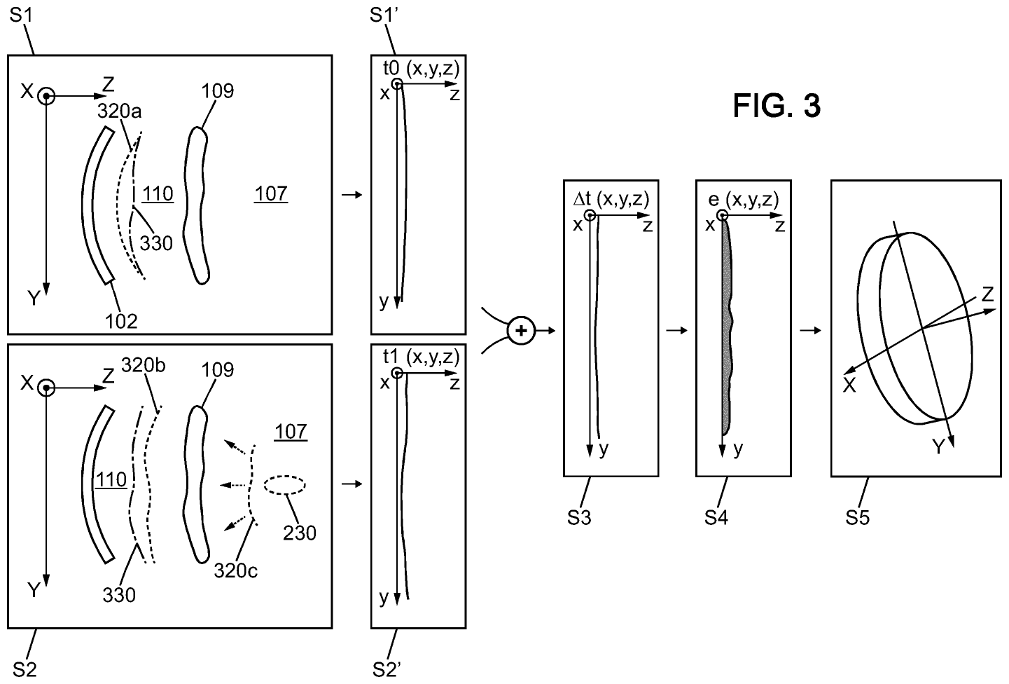

In all cases, it is necessary to be able to generate, with the greatest precision possible, one or more predetermined objective ultrasonic wave field(s) in the brain 107 of the patient P, for example for focusing the ultrasonic waves emitted by an ultrasound probe 102 on one or more points of the brain, or for generating more complex wave fields. As shown in FIG. 2 (or FIG. 4), the objective wave field may be, for instance, a wave field focused on a focal spot F in the brain 107, or possibly a plurality of focal spots, or one or a plurality of more complex three-dimensional shape.

The mechanism 100 may comprise an ultrasound probe 102 and a control system 111. The ultrasound probe 102 may comprise an emission surface and may be configured to transmit ultrasound waves 210 into the homogeneous internal part 107 (i.e. brain of the patient P) comprised in the medium 103. The ultrasound waves may be generated by one transducer 203 (or ultrasound transducer) or a plurality of transducers (or a plurality of ultrasound transducers) comprised in the ultrasound probe 102.

In one or several embodiments, the plurality of transducers may comprise a number of transducers comprise between 1 and 128 transducers.

According to an example, the transducer or the plurality of transducers 203 may have a circular shape defined by a diameter comprised between 10 and 400 millimeters and present a radius of curvature which may be comprised between 5 and 200 millimeters. According to one example, the transducer may have curvature radius of 59 millimeters for an aperture of 67 millimeters (at 500 kHz).

In one or several embodiments, the ultrasound probe may be a single transducer, and which may be a focused or unfocused transducer.

Furthermore, the control system may be programmed (or configured) such that the ultrasonic waves may be transmitted at a rate more than 100 ultrasonic waves per second, for instance hundreds to several thousands of ultrasonic waves per second. The control system may for instance include a control unit 111a and a computer 111b. In this example, the control unit 111a may be used for controlling the ultrasound probe 102, while the computer 111b may be used for controlling the control unit 111a, determining the zone to treat in the internal part 107, and design the acoustic lens 109 for instance. In a variant, a single electronic device could fulfill all the functionalities of control unit 111a and computer 111b.

The mechanism 100 may be comprised of an acoustic lens 109 (or self-positioning acoustic lens) which may be interposed between the ultrasound probe 102 and a surface of the medium, for instance the skull 108 of the patient P. The acoustic lens may comprise a front surface 109a and a back surface 109b.

In one or several embodiments, the ultrasound probe 102 may be mechanically constrained by 102a with the acoustic lens 109 (i.e. the self-positioning acoustic lens) so that the emission surface of the ultrasound probe may face the back surface 109b of the acoustic lens at distance comprised between 0 and 150 millimeters. According to an example, the ultrasound probe may be fixed with the edges of the acoustic lens by using rod 102a (e.g. in metal or polymer), and the set (acoustic lens with the ultrasound probe) may be maintained by elastic means on the head of the patient P during the emission of ultrasound waves. According to another example, the set may be maintained by the hand of the patient or of an operator during the emission of ultrasound waves or maintained by using a robotic arm (piloted manually or automatically by an operator for instance).

According to another example, ultrasound probe 102 may be, for instance integrated in a helmet (not shown) positioned in a predetermined way on the head 103 of the patient P, or else said ultrasound probe 102 may be carried by any other known positioning system.

In all cases, the ultrasound probe 102 may be positioned outside of the skull 108 of the patient in a predetermined position. Such predetermined position of the ultrasound probe may be the position near the zone to treat at the focal spot 230 (or focal point).

A gel or liquid, which could be contained in a flexible pouch (not shown), may be interposed between the acoustic lens 109 and the skull 108 and/or between the ultrasound probe 102 and the acoustic lens 109, i.e. between the front surface 109a and the skull 108, so as to assure good transmission of ultrasonic waves. Here, this gel or liquid will be called external medium and considered as being part of the propagation medium 103 of the ultrasonic waves.

The acoustic lens 109 (or self-positioning acoustic lens) may be made of any material in which the speed of compressive acoustic waves c is different from the speed c of said waves in water (c is about 1480 m/s at 20° C.). The acoustic lens may, for example, be made of silicone, in particular polydimethylsiloxane, also called PDMS (c_{I}=1030 m/s) or the Elite Double products (Zhermack SpA, Italy), or of plastic, in particular polymethylpentene, known under the name TPX® (c_{I}=2090 m/s).

Some parts of the acoustic lens could be made of an attenuating material, reflecting and/or absorbing ultrasonic waves, in particular for locally attenuating the ultrasonic wave field made in the brain 107 or the skull 108, for example for enhanced beam shaping, and/or to limit the heating of the skull or to limit the insonification volume in the brain 107.

In reference to FIG. 2, the front surface 109a of the acoustic lens may be in physical contact with the outer surface 108a of the medium 103. In general, the outer surface of the medium is not perfectly spherical, so that a plurality of specific areas are presents on the surface of the medium.

For instance, the surface of the medium 103 may be limited to a specific area (or a restricted and specific area) of the surface of the medium in regard to a zone in the internal part 107 (e.g. zone to treat) where it is wanted to generate the objective wave field comprising one or a plurality of focal spots or one or a plurality of more complex three-dimensional shape. This surface of the medium (on FIG. 2) limited to a specific area may have a specific shape existing only in this specific area allowing thus a self-positioning of the acoustic lens at the surface of the medium. For instance, this specific shape may be a specific shape (or a restricted and specific area) of the skin on top of the skull existing only in this specific area at the surface of the medium. Indeed, the aspherical shape of the head may present different local configurations along the surface of the head.

As described previously, when focusing ultrasound waves in a homogeneous internal part 107 such as the brain in the medium 103, with one transducer only or few transducers (less than 128 transducers), the presence of a barrier such as the skull bone may disrupt the focus of the ultrasound waves leading to the deformation of the focal spot 230 (or the plurality of focal spots or one or a plurality of more complex three-dimensional shape). The deformation of the focal spot may be problematic when it is wanted to treat a specific zone (or precise zone) in the internal part 107 such a brain. The document WO2017001781A1 describes a method for designing and fabricating an ultrasound lens suitable to focus ultrasound waves in a medium such as a brain (i.e. located behind a bone barrier) and compensating, partially or totally, the deformation of the focal spot. In this purpose, the method of conception of the ultrasound lens described in WO2027001781A1 is to determine a thickness “e(x,y)” from the back surface to the front surface of the ultrasound lens in order to compensate the phase delays induced by the barrier. No constrain is imposed on the front surface of the lens in the document WO2017001781A1: only the thickness of the lens is constrained. According to the document WO2017001781A1, it may allow, when an ultrasound wave 210 is generated by the ultrasound probe 102 (comprising at least one transducer 203) and crossing the ultrasound lens, to shape the ultrasound wave (by phasing out) in order to compensate the delays caused by the barrier and thus to restore the ultrasound wave into the internal part as if the barrier was inexistent or negligible, and avoiding, therefore, the deformation of the focal spot 230 (or focal point). However, such acoustic lens described in the document WO2017001781A1 is not self-positioning, and its use in the treatment of specific zone in the brain still requires the use of a neuronavigation system or any other navigation device.

In reference to the FIG. 2, it may be possible to determine a geometry of the acoustic lens (or self-positioning acoustic lens) comprising a thickness “e(x,y,z)” from the front surface 109a to the back surface 109b of the acoustic lens and specific shape of the front surface for instance. The acoustic lens of the FIG. 2 having a determined acoustic lens thickness and a front surface fitting with only one specific area on the outer surface of the medium, allowing a self-positioning on the surface of the medium, may allow to obtain one or a plurality of focal spots or one or a plurality of more complex three-dimensional shape in a specific zone of the internal part 107 such as a brain when transmitting a predetermined ultrasound wave without using any neuronavigation system.

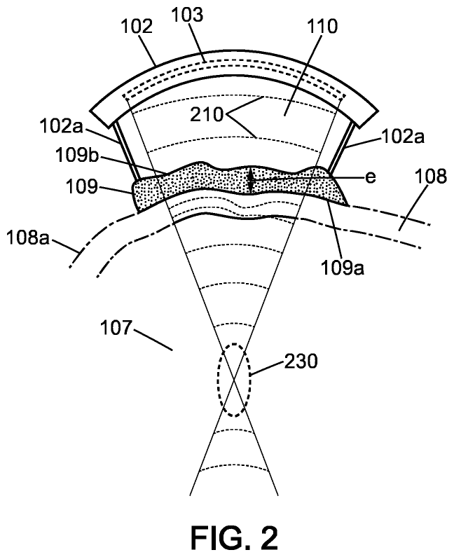

FIG. 3 illustrates the method for obtaining a self-positioning acoustic lens such as the acoustic lens of the present disclosure.

The method for obtaining such acoustic lens may be obtained as follow.

### (a) Determination of a Three-Dimensional Model of the Medium 103

In advance, a three-dimensional model of the medium 103 may be determined comprising a mapping of the acoustical properties in the medium 103 comprising the homogeneous internal part 107 and the barrier 108. For instance, the internal part 107 may be the brain of the patient P and the barrier 108 may be the skull bone of the patient P.

This step may generally comprise an imaging operation, for instance by computed tomography (CT) or by Magnetic resonance imaging (MRI, with conventional imaging or by ultras-short echo-time imaging) or by Ultrasound imaging, in order to determine the acoustic properties of the barrier 108. For instance, the information obtained by imaging the medium 103 may be used to determine in particular the mass density ρ, the speed of sound c and/or the absorption coefficient τ of said ultrasonic waves, at each point of the aberrating barrier 108 and of the medium 107.

The three-dimensional model may, for instance, be loaded into the computer 111b. A user may position the desired ultrasonic wave field, for example one or more focal spot(s) (or focal point(s)) 230, on the image of the medium 107 (FIG. 2). This positioning may, for instance, be done by viewing a target to be treated or other zone(s) of interest on the image of the medium 107 and by locating this zone on the image using the user interface of the computer 111b (for example mouse, touchscreen or other).

In the following, for sake of simplicity, “the zone of interest” will be referred to for designating one or more focal spots or one or a plurality of more complex three-dimensional shape.

### (b) Simulation

The propagation of an ultrasonic wave through the aberrating barrier 108 may be simulated from the aforementioned three-dimensional model for next determining the delays or phase shifts which will have to be induced by the acoustic lens (or self-positioning acoustic lens), and then deducing from that the geometry (or shape) to give to the acoustic lens, as for instance, the distance between the front surface and the back surface of the acoustic lens (i.e. the thickness of the acoustic lens). This simulation step may be done by the aforementioned computer 111b or another computer.

In one or several embodiments, this simulation step may comprise the following sub-steps:

### (b1) Simulation of the Back Propagation of the Desired Ultrasonic Wave in the Zone of Interest in the Three-Dimensional Model (S2)

As shown in S2 in FIG. 3, during this sub-step b1, a back propagation of the predetermined objective ultrasonic wave field 320c, in the medium 107 is simulated from the focal spot 230 (i.e. zone of interest) in the medium 107 to a single control surface 330.

The predetermined control surface 330 may have a shape which may be shaped as the shape of the outer surface of the medium 107, i.e. the shape of the surface of the skin surrounding the skull (i.e. the shape of the surface of the barrier 108) in a specific area. This specific area may be the area of the surface of the medium which may be close to the focal spot 230 and on which the acoustic lens (or self-positioning acoustic lens) is intended to be positioned.

The shape of the predetermined control surface 330 may have been determined in advance by using the information obtained from the imaging operation at the step (a) previously presented.

This simulation may be done, for example, by the computer 111b, by using a wave equation such as equation (1) below:

$$( 1 + τ ( \vec{r} ) \frac{\partial }{\partial t} \cdot ) [ ρ ( \vec{r} ) ∇ \cdot ( \frac{1}{ρ ( \vec{r} )} ∇ p ( \vec{r} , t ) ) ] - \frac{1}{{c ( \vec{r} )}^{2}} \frac{{\partial }^{2} p ( \vec{r} , t )}{\partial {t}^{2}} = S ( \vec{r} , t ) , \qquad ( 1 )$$

Where \vec{r} designates a position vector for the point considered; p designates the pressure; and S designates the ultrasonic signals generated by an ultrasonic source which could be present at the point considered.

The propagation of the ultrasonic waves in the aberrating barrier 108 and the internal part 107 may be simulated in the computer by finite differences, by discretizing the equation (1) above. The simulation may also be done by finite elements, or by an impulse diffraction method, or any other known method.

The simulation may be performed by the control unit or the computer of the control system or by the single electronic device or by others electronic device which may include a processor, a storage and an instruction set configured to implement the simulation.

### (b2) Determination of the First Arrival Times on a Control Surface 330 (S2′)

As shown in S′2 in FIG. 3, the arrival times t₁(x, y, z) of the ultrasonic wave 320c, 320b representative of the back propagation of the predetermined objective wave field may be determined at control points M(x, y, z) preferably belonging to a single control surface 330 located at the outer surface of the medium 103, for instance confused with the surface 108a.

The arrival times t₁(x, y, z) may be counted, for example, from the instant of the emission of the ultrasonic wave 320c in said zone 230, where x, y and z are respectively the coordinates on the axes X, Y and Z of each control point M considered. The arrival times t₁(x, y, z) are hereafter be called first arrival times.

### (b3) Determination of the Second Arrival Times on the Control Surface 330 (S1-S1′)

As shown in S1 and S′1 on FIG. 3, it may be possible to determine arrival times t₀(x, y, z) of a predetermined ultrasonic wave 320a emitted by an ultrasound probe 102 comprising at least one transducer, at different control points M(x, y, z), preferably belonging to the single control surface 330 located at the outer surface of the medium 103.

As for (b1), the predetermined control surface 330 may be shaped according to the shape of the surface of the medium, i.e. the shape of the surface of the skin surrounding the skull in a specific area. This specific area may be the area of the surface of the medium which may be close to the focal spot 230 and on which the acoustic lens (or self-positioning acoustic lens) is intended to be positioned.

The arrival times t₀(x, y, z) may be determined by the computer 111b or other (up to an arbitrary constant time T′₀ common to the entire control surface 330), because the external part of the medium (outside of the aberrating barrier) is considered as homogeneous for the propagation of ultrasonic waves.

In the simple and the most common case where the ultrasound probe (or the transducer) is spherical with radius R, this determination may be done very simply in the form:

$${t}_{0} ( x , y , z ) = {T}_{0}^{′} - \frac{d ( M )}{c}$$

where:
d(M) is the distance between the control point M(x, y, z) and the center of curvature of the ultrasound probe 109;
c is the speed of sound in the external part of the coupling medium 110.

More generally, the arrival times t₀(x, y, z) may be determined by calculation of the ultrasonic wave propagation between the ultrasound probe and the control points or by any other known wave propagation method, which is simple in a homogeneous medium (the propagation can then be done in a simplified model allowing an analytic calculation).

The arrival times t₀(x, y, z) will hereafter be called second arrival times.

### (b4) Determination of the Delay Law of the Lens (S3)

As shown in S3 on FIG. 3, the delay law Δt₀(x, y, z) may be determined at various control points M(x, y, z), equal to the sum of the first arrival times t₁(x, y, z) and second arrival times t₀(x, y, z)

### (c) Calculation of the Acoustic Lens (S4)

Starting from the aforementioned delay law Δt₀(x, y, z) and the shape of the front surface (comprised in the geometry of the self-positioning acoustic lens) which has to match with the outer surface of the medium or with a specific area of the outer surface of the medium, the computer 111b or other may next calculate the profile of the acoustic lens 109 which, when it is interposed between the ultrasound probe 102 and the aberrating barrier 108 and positioned on the outer surface of the medium, may be able to add said delay Δt₀(x, y, z), such that when the ultrasound probe 102 emits the predetermined ultrasonic wave 320a, the predetermined wave after traversing the acoustic lens and then the aberrating barrier may produce the predetermined ultrasonic wave field in the zone of interest, corresponding to one or a plurality of focal spots or one or a plurality of more complex three-dimensional shape 230 in the internal part 107.

More precisely, the thickness e of the acoustic lens 109, i.e. the thickness between the front surface and the back surface of the acoustic lens, may be calculated at each point of the acoustic lens corresponding to a control point M(x, y, z) of the predetermined control surface 330, such that the thickness e(x, y, z) of the acoustic lens may introduce an ultrasonic wave propagation time correction corresponding to said delay law modulo the period T=1/f of the ultrasonic wave (where f is the central frequency of the ultrasonic wave) and/or up to an arbitrary constant time To common to the entire control surface.

In one or several examples (like the case where the ultrasound probe is a focused transducer or a single focused transducer), the delay Δt₀(x, y, z) may be defined in spherical coordinates according to Δt₀(r, θ, φ) with the center of coordinates located at the center of curvature of the transducer 102, and may be used to calculate the spherical coordinates of each point L(r′, θ, φ) of the back surface of the acoustic lens in regard to a corresponding control point M(r, θ, φ), with the following formula (3):

$${r}^{′} = r + \frac{Δ t ( r , θ , φ )}{\frac{1}{c} - \frac{1}{{c}_{1}}} + {e}_{0} \qquad ( 3 )$$

Where c is the speed of the ultrasonic wave in the coupling medium 110 outside the aberrating barrier 108 and c₁ is the speed of the ultrasonic wave in the lens.

The constant e₀ is a real number such that the thickness of the lens is positive at each point and greater than a minimum value necessary to ensure the robustness of the lens, said minimum value depending on the material of the lens.

In the case of the emission of a monochromatic wave, in one or several embodiments, the spherical coordinates of each point L(r′, θ, φ) of the back surface of the acoustic lens in regard to the corresponding control point M(r, θ, φ), may be calculated with the following formula (4):

$${r}^{′} = r + \frac{Δ t ( r , θ , φ )}{\frac{1}{c} - \frac{1}{{c}_{1}}} + {e}_{0} , modulo \frac{T}{\frac{1}{c} - \frac{1}{{c}_{1}}} \qquad ( 4 )$$

Modulo

$$\frac{T}{\frac{1}{c} - \frac{1}{{c}_{1}}}$$

means that

$${r}^{′} = r + \frac{Δ t ( r , θ , φ )}{\frac{1}{c} - \frac{1}{{c}_{1}}} + {e}_{0} + n \cdot \frac{T}{\frac{1}{c} - \frac{1}{{c}_{1}}} ) ,$$

where n is a positive or negative integer such that r′−r has a positive value. The integer n may be common to the entire acoustic lens 109 (simple lens) or could be different according to the regions the acoustic lens 109 (for instance in the case of the design of a Fresnel lens).

These spherical coordinates of each point L(r′, θ, φ) of the back surface of the acoustic lens in regard to the corresponding control point M(r, θ, φ) may be used to compute the distance between the front and the back surface corresponding to the thickness of the acoustic lens (or self positioning acoustic lens).

It will be noted that the lens calculation step (c) may consider at least the travel times of the predetermined ultrasonic wave in the acoustic lens 109 and the angles of incidence and refraction of said predetermined ultrasonic wave with the surface of the acoustic lens 109. The lens calculation step (c) may also consider the entire propagation of said predetermined ultrasonic wave, including refraction by each surface of the self-positioning acoustic lens and/or echoes between the ultrasound probe 102 comprising at least one transducer, the acoustic lens 109 and the aberrating barrier 108 and/or including echoes within the self-positioning lens. The lens calculation step (c) may be done iteratively in order to optimize the ultrasonic energy in the zone of interest, using an analytic or numerical model.

The calculation step may be carried out by a calculation device which may be the control unit or the computer of the control system or by the single electronic device or by others electronic device which may include a processor, a storage and an instruction set configured to implement the calculation step.

### (d) Implementation of the Acoustic Lens

Then, once the geometry of the self-positioning acoustic lens, comprising the shape of the front surface and the distance (i.e. the thickness of the acoustic lens) between the front and back surface, is determined, the acoustic lens may be produced by any rapid production method such as:
Three-dimensional printing of the acoustic lens 109;
Numerically controlled machining of a block of material in order to form the acoustic lens 109;
A process with two sub-steps comprising:
   (d1) A sub-step of mold production (not shown) done by a method chosen among three-dimensional printing of the mold and/or numerically controlled machining of a block of material for forming the mold;
   (d2) A sub-step of molding in which said lens 109 is molded in said mold.

In all scenarios, the acoustic lens may be produced in a material having mechanical characteristics (in particular hardness), and therefore acoustic wave propagation characteristics, modifiable by exposure to a predetermined radiation.

The predetermined radiation may be, for instance ultraviolet radiation.

The material in question may be for instance silicone, in particular polydimethylsiloxane, also called PDMS (c_{I}=1030 m/s) or the Elite Double products (Zhermack SpA, Italy),, or plastic, like for example polyvinylmethylsiloxane (PVMS), which hardens under ultraviolet radiation.

In this case, during lens calculation step (c), the acoustic lens 109 may be calculated by determining the local acoustical properties of the acoustic lens 109 with which to achieve the desired objective wave field. These mechanical properties can be determined in addition to the shape of the acoustic lens 109, or as a variant, the objective wave field could be obtained just by local variations of local propagation characteristics of the acoustic lens 109.

During the step (d) of implementation of the acoustic lens 109, said lens is locally exposed to said predetermined radiation to obtain the local acoustical properties determined during the lens calculation step (c).

### (e) Use

Once the acoustic lens 109 is produced, it may be used to treat a specific zone in internal part such as the brain. For this purpose, the acoustic lens 109 may be positioned on the skin of the patient P so that the front surface of the acoustic lens is in physical contact with the surface of the skin and the shape of the front surface of the acoustic lens may fit in complementary way the shape of the surface of the medium in only one possibility. More precisely, the shape of the front surface of the acoustic lens may match with only one specific area of the surface of the skull so that the acoustic lens is self-positioned.

Because the acoustic lens being self-positioned, the patient P may be positioning himself the acoustic lens on which the ultrasound probe is fixed since only one position is possible and corresponds to the position allowing to treat the right zone in the brain. For instance, the patient may place the self-positioning acoustic lens on his head with his hand and wait for instructions from operators before activating the ultrasound probe. In one or several alternatives, the acoustic lens on which the ultrasound probe is fixed (in a constrained way) may be placed with a robotic arm, manually or automatically, or be located in a helmet, or be held by an operator.

An operator may then send the predetermined wave 320a from the ultrasound probe 102 through the self-positioning acoustic lens 109.

The insonification method may be carried out by the control unit or the computer of the control system or by the single electronic device. For instance, the operator may activate the insonification method by using the control system.

As shown in FIG. 4, the self-positioning acoustic lens 109, for which the front surface is in physical contact with the surface of the skin in a specific area, may induce aberration corrections which form the wavefront 320b upstream from the skull 108, such that after passage through the skull 108 (or another aberrating barrier), the wavefront 320c is corrected from the aberrations induced by the skull (or another aberrating barrier). Thus, the objective wave field may result, for example in the focal spot 230.

Thus, the wave sent into the brain or other internal part 107 may serve to treat a zone of interest (or specific zone) 230 as previously explained, but without using any neuronavigation system. Indeed, thanks to the configuration of the acoustic lens as the acoustic lens thickness and the fact that the front surface is shaped for only one specific area of the surface of the medium (for instance the surface of the skin surrounding the skull), the acoustic lens is configured to be placed at only one possible position, therefore self-positioning, on the skin allowing to generate the focal spot 230 (or a plurality of focal spots or one or a plurality of more complex three-dimensional shape) comprising the correct zone to treat without using any neuronavigation system. As the acoustic lens is designed for a specific zone to treat in the brain from only one possible position on the skull, the risk of mistakes (e.g. treat the wrong zone in the brain) is highly minimized.

In addition, the brain or another internal part 107 at least in the neighborhood around the zone of interest comprised in the focal spot 230 could be imaged, when the ultrasound probe 102 comprises ultrasonic transducers for imaging, because the acoustic lens 109 may compensate the aberrations due to the skull or other aberrating barrier 108 both during transmitting and receiving.

The method according to the present disclosure may further comprise an optional step (f) of verification of the positioning of the device, during which ultrasonic echoes which may be reflected on said aberrating barrier are recorded on the ultrasound probe and the echoes are compared to the same reflected signal simulated by using the aforementioned model of the medium 103.

The control unit and/or the computer or/and the single electronic device may include a processor, a storage (e.g. a memory) and an instruction set configured to implement the methods according to the present disclosure. The control unit and/or the computer or/and the single electronic device may include an input interface and an output interface. In variant, the computer may replace the control unit, or the control unit may replace the computer. In a variant, a single electronic device could fulfill all the functionalities of the control unit and the computer.

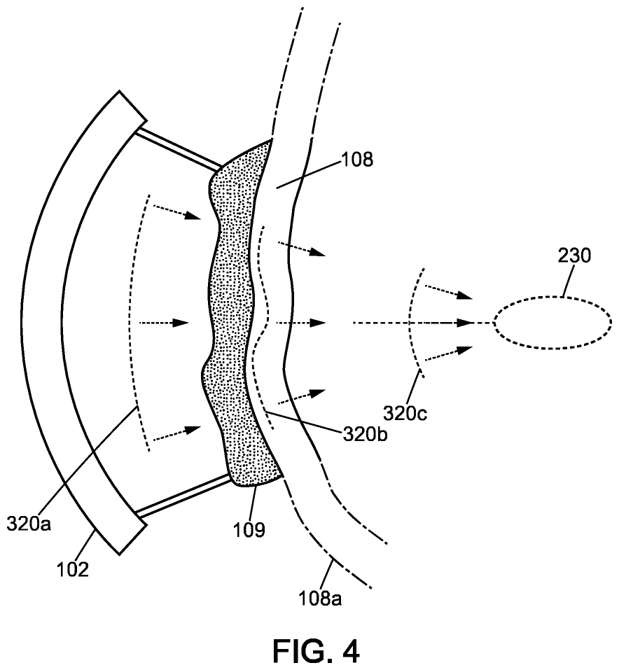

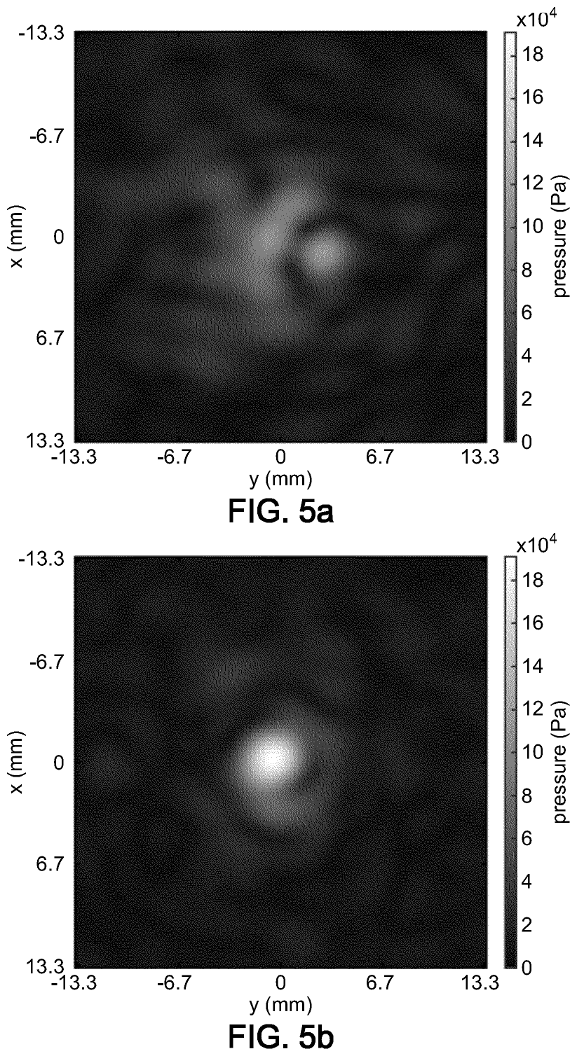

FIGS. 5a and 5b illustrates the effects on the predetermined ultrasound wave field generated when a self-positioning acoustic lens is used. More precisely, FIG. 5a illustrates a predetermined ultrasound wave field in an area which is not corrected, and FIG. 5b illustrates a predetermined ultrasound wave field in the same area corrected with a self-positioning acoustic lens.

In the experimental setup allowing to obtain the above predetermined ultrasound wave field, a self-positioning acoustic lens was designed and manufactured for a cadaver human skull, according to the method presented previously. The front surface of the self-positioning acoustic lens was shaped to be constrained and shaped to match the surface of the outer surface of the skull. The back surface of the self-positioning acoustic lens was also shaped to compensate for skull aberrations using structural and mechanical information derived from a CT scan. The experimental setup also comprises a water tank, an ultrasound transducer and a hydrophone. The acoustic pressure field generated by the transducer is measured by the hydrophone.

Two acoustic pressure fields are recorded. One after propagation through the human skull without the self-positioning acoustic lens (FIG. 5a) and one after propagation through the self-positioning acoustic lens and the human skull (FIG. 5b).

These data show that a self-positioning acoustic lens can both match the outer surface of an aberrating barrier to allow the positioning of the lens and also correct for the aberrations induced by the skull for a precise focusing of the wave.

1. A self-positioning acoustic lens comprising a front surface and back surface, said back surface being opposed to the front surface, said self-positioning acoustic lens being adapted for transmitting an ultrasound wave into a medium comprising at least one aberrating barrier and a substantially homogeneous internal part masked by said aberrating barrier, said ultrasound wave being generated by an ultrasound probe located outside of the medium and said back surface facing the emission surface of the ultrasound probe,
wherein the self-positioning acoustic lens being configured to, when the self-positioning acoustic lens is interposed between the ultrasound probe and the aberrating barrier and when the ultrasound probe transmits a predetermined ultrasonic wave, generate a predetermined objective ultrasonic wave field in at least one predetermined area belonging to said internal part despite the presence of the aberrating barrier,
and wherein the front surface is constrained and is adapted to match the outer surface of the medium in order to be self-positioned on the outer surface of the medium, said outer surface of the medium not being perfectly spherical, and wherein the back surface being spaced from the front surface according to a specific self-positioning acoustic lens thickness in order to create the said predetermined objective ultrasonic wave field.

2. Self-positioning acoustic lens according to claim 1 wherein the surface of contact between the front surface of the self-positioning acoustic lens and the outer surface of the medium is limited to a restricted and specific area on the outer surface of the medium.

3. Self-positioning acoustic lens according to claim 1 wherein the aberrating barrier is a skull and the outer surface is the skin surrounding the skull, and the restricted and specific area corresponds to a part of the outer surface in contact with the front surface of the self-positioning acoustic lens.

4. Self-positioning acoustic lens according to claim 1 wherein the thickness of the self-positioning acoustic lens from each point of the front surface of the self-positioning acoustic lens satisfies the following formula:
$$e ( x , y , z ) = \frac{Δ t ( x , y , z )}{\frac{1}{C} - \frac{1}{{C}_{1}}} + {e}_{0}$$
where:
c is the speed of the ultrasonic wave in coupling medium outside of the aberrating barrier;
c₁ is the speed of the ultrasonic wave in the self-positioning acoustic lens;
e₀ is a real number such that e(x, y, z) has a value at each point of the self-positioning acoustic lens that is positive and greater than a minimum value necessary to ensure the robustness of the lens, said minimum value depending on the material of the self-positioning acoustic lens; and
Δt₀(x, y, z) is a delay law calculated to recreate the predetermined

5. Self-positioning acoustic lens according to claim 1 wherein when the ultrasound probe is a focused transducer, the thickness is defined so that, in spherical coordinates, each point L(r′, θ, φ) of the back surface of the self-positioning acoustic lens in regard to a corresponding point on the front surface M(r, θ, φ), verifies the following formula:
$${r}^{′} = r + \frac{Δ t ( r , θ , φ )}{\frac{1}{C} - \frac{1}{{C}_{1}}} + {e}_{0}$$
where:
c is the speed of the ultrasonic wave in the medium outside of the aberrating barrier;
c₁ is the speed of the ultrasonic wave in the self-positioning acoustic lens;
e₀ is a real number such that the thickness of the self-positioning acoustic lens is positive at each point and greater than a minimum value necessary to ensure the robustness of the self-positioning acoustic lens, said minimum value depending on the material of the self-positioning acoustic lens; and
Δt₀(r, θ, φ) is a delay law calculated to recreate the predetermined objective despite the presence of the aberrating medium, said delay law is calculated at each point M(r, θ, φ) on the front surface defined in the spherical coordinates with the origin of the spherical reference system being located at the center of curvature of the focused transducer.

6. Self-positioning acoustic lens according to claim 1 wherein the front surface is constrained and is adapted to match the outer surface of the medium in a complementary way.

7. A production method for implementing a self-positioning acoustic lens comprising a front surface and back surface, said back surface being opposed to the front surface, said self-positioning acoustic lens being adapted for transmitting an ultrasound wave into a medium comprising at least one aberrating barrier and a substantially homogeneous internal part masked by said aberrating barrier, the ultrasound wave being generated by an ultrasound probe located outside of the medium and said back surface being adapted to face the emission surface of the ultrasound probe;
the production method comprising at least:
imaging the medium for generating a mapping of the acoustic properties of the medium;
using a model to estimate time delays at a given control surface comprised within an expected volume of the self-positioning acoustic lens, said time delays allowing to produce predetermined objective ultrasonic wave field in at least one predetermined area belonging to said internal part;
calculating a geometry of the self-positioning acoustic lens comprising at least a determination of a first shape of front surface and a determination of a thickness between the front and the back surface, by using the model of the medium comprising the mapping of acoustic properties, the self-positioning acoustic lens being configured to generate the time delays so that, when the self-positioning acoustic lens is interposed between the ultrasound probe and the aberrating barrier and when the ultrasound probe transmits a predetermined ultrasonic wave it produces the predetermined objective ultrasonic wave field in at least one predetermined area belonging to said internal part despite a presence of the aberrating barrier;
implementing the self-positioning acoustic lens by using the calculated self-positioning acoustic lens; and
wherein the first shape of the front surface of the self-positioning acoustic lens is determined to match the outer surface of the medium in order to be self-positioned on the outer surface of the medium, said outer surface of the medium not being perfectly spherical.

8. (canceled)

9. The production method according to claim 7 wherein the calculation of the geometry of the self-positioning acoustic lens is preceded by a simulation comprising:
(b1) a back-propagation in the medium of said predetermined objective ultrasonic wave field from the predetermined area to a chosen position of the control surface is simulated;
(b2) first arrival times t₁(x, y,z) of said back-propagation of said predetermined objective ultrasonic wave field to said chosen position of the control surface are determined;
(b3) a propagation in a coupling medium of the ultrasonic wave emitted by the ultrasound probe disposed outside of the medium to the chosen position of the front face of the self-positioning acoustic lens is calculated, and second arrival times t₀(x, y, z) of said ultrasonic wave to said chosen position of the front face of the self-positioning acoustic lens are determined;
(b4) a delay law Δt₀(x, y, z) at said chosen position of the control surface equal to the sum of the first arrival times t₁(x, y, z) and second arrival times t₀(x, y, z) is determined,
wherein the geometry of the self-positioning acoustic lens is calculated using said delay law Δt₀(x, y, z), so that when the ultrasound probe emits the ultrasonic wave through said self-positioning acoustic lens, said predetermined ultrasonic wave reproduces the objective wave field in said predetermined area after passing through said aberrating barrier, and
wherein control points M(x, y, z) distributed on the front surface of the self-positioning acoustic lens and at which points the second arrival times t₀(x, y, z) and the first arrival times t₁(x, y, z) are determined, belong to a single control surface, preferably the control surface is located at the outer surface of the medium.

10. The production method according to claim 9, wherein when the ultrasound probe is a focused transducer, the thickness is determined so that, in spherical coordinates, each point L(r′, θ, φ) of the back surface of the self-positioning acoustic lens in regard to a corresponding point on the front surface M(r, θ, φ), is calculated with the following formula:
$${r}^{′} = r + \frac{Δ t ( r , θ , φ )}{\frac{1}{C} - \frac{1}{{C}_{1}}} + {e}_{0}$$
where:
c is the speed of the ultrasonic wave in the coupling medium of the aberrating barrier;
c₁ is the speed of the ultrasonic wave in the self-positioning acoustic lens;
e₀ is a real number such that the thickness of the self-positioning acoustic lens is positive at each point and greater than a minimum value necessary to ensure the robustness of the self-positioning acoustic lens, said minimum value depending on the material of the self-positioning acoustic lens;
Δt₀(r, θ, φ) is a delay law calculated to recreate the predetermined objective despite the presence of the aberrating medium, said delay law is calculated at each point on the front surface M(r, θ, φ) defined in the spherical coordinates with the origin of the spherical reference system being located at the center of curvature of the focused transducer.

11. The production method according to claim 9, wherein the thickness e(x, y, z) is calculated at each point M(x, y, z) of the front surface with the following formula:
$$e ( x , y , z ) = \frac{Δ t ( x , y , z )}{\frac{1}{C} - \frac{1}{{C}_{1}}} + {e}_{0}$$
where:
c is the speed of the ultrasonic wave in the coupling medium outside of the aberrating barrier;
c₁ is the speed of the ultrasonic wave in the self-positioning acoustic lens;
e₀ is a real number such that the thickness e(x, y, z) of the self-positioning acoustic lens is positive at each point and greater than a minimum value necessary to ensure the robustness of the self-positioning acoustic lens, said minimum value depending on the material of the self-positioning acoustic lens.

12. The production method according to claim 7 wherein implementing the self-positioning acoustic lens is done by a method chosen among three-dimensional printing of the self-positioning acoustic lens and/or digitally controlled machining of at least one block of material for forming the self-positioning acoustic lens.

13. The production method according to claim 7 wherein a calculation of geometry of the self-positioning acoustic lens considers the entire propagation of said predetermined ultrasonic wave, including refraction by each surface of the self-positioning acoustic lens and/or echoes between the ultrasound probe, the self-positioning acoustic lens and the aberrating barrier and/or including echoes within the self-positioning acoustic lens.

14. (canceled)

15. An insonification method of a medium comprising at least one aberrating barrier and a substantially homogeneous internal part masked by said aberrating barrier, said outer surface of the medium not being perfectly spherical, the insonification method comprising a manufacturing of a self-positioning acoustic lens according to claim 7;
the insonification method further comprises:
transmitting said predetermined ultrasonic wave through the self-positioning acoustic lens, the ultrasound wave being generated by said ultrasound probe located outside of the medium and said back surface being adapted to face the emission surface of the ultrasound probe,
and wherein the self-positioning acoustic lens being disposed on the outer surface of the medium so that the first shape of the front surface of the self-positioning acoustic lens match with the outer surface of the medium in order to be self-positioned on the outer surface of the medium.

16. The insonification method according to claim 15, wherein the surface of contact between the front surface of the self-positioning acoustic lens and the outer surface of the medium is limited to a restricted and specific area on the outer surface of the medium.

17. The insonification method according to claim 15 wherein the medium in which the ultrasonic waves propagate is a human or animal head, where the aberrating barrier is a skull and the internal part is a brain.

18. A method for calculating a geometry of a self-positioning acoustic lens, the self-positioning acoustic lens being suitable for insonification of a medium comprising at least one aberrating barrier and a substantially homogeneous internal part masked by said aberrating barrier, the outer surface of the medium not being perfectly spherical,
the method for calculating the geometry of the self-positioning acoustic lens, by using a model of the medium comprising a mapping of acoustic properties, the self-positioning acoustic lens being configured to, when the self-positioning acoustic lens is interposed between the ultrasound probe and the aberrating barrier and when the ultrasound probe transmits a predetermined ultrasonic wave, generates a predetermined objective ultrasonic wave field in at least one predetermined area belonging to said internal part.

19. A calculation device of a geometry of a self-positioning acoustic lens, the self-positioning acoustic lens being suitable for insonification of a medium comprising at least one aberrating barrier and a substantially homogeneous internal part masked by said aberrating barrier, the outer surface of the medium not being perfectly spherical,
the calculation device being configured to calculate said geometry of the self-positioning acoustic lens, using a model of the medium comprising the mapping of acoustic properties, the self-positioning acoustic lens being configured to, when the self-positioning acoustic lens is interposed between the ultrasound probe and the aberrating barrier and when the ultrasound probe transmits a predetermined ultrasonic wave, generates a predetermined objective ultrasonic wave field in at least one predetermined area belonging to said internal part.

20. A device for manufacturing a self-positioning acoustic lens, comprising a calculation device of a self-positioning acoustic lens according to claim 18 and a manufacturing device producing the self-positioning acoustic lens.

21. A computer program comprising instructions which, when the program is executed by a computer comprising a processor and a storage, cause the computer to carry out the method of claim 7.

22. A computer-readable medium having stored thereon the computer program of claim 21.
